(19) United States
(12) Patent Application Publication
(10) Pub. No.: US 2017/0100484 A1
(43) Pub. Date: Apr. 13, 2017

(54) DRUG DELIVERY CARRIER FOR SUSTAINED RELEASE OF MEDICINAL PROTEINS AND METHOD FOR PRODUCTION THEREOF

(71) Applicant: KANGWON NATIONAL UNIVERSITY UNIVERSITY-INDUSTRY COOPERATION FOUNDATION, Chuncheon-si (KR)

(72) Inventors: Jin Chul KIM, Chuncheon-si (KR); Ju Hyup LEE, Chungcheongnam-do (KR); Kyeong Nan KWON, Gangneung-si (KR); Dong Youl YOON, Seoul (KR); Hong ZHANG, Chuncheon-si (KR)

(73) Assignee: KANGWON NATIONAL UNIVERSITY UNIVERSITY-INDUSTRY COOPERATION FOUNDATION, Chuncheon-si (KR)

(21) Appl. No.: 15/067,151

(22) Filed: Mar. 10, 2016

(30) Foreign Application Priority Data
Oct. 7, 2015 (KR) 10-2015-0141034

(51) Int. Cl. A61K 47/36; A61K 47/12; A61K 47/10

(52) U.S. Cl. CPC A61K 47/36; A61K 47/10; A61K 47/12

(57) ABSTRACT

Disclosed is a drug delivery carrier for sustained release of medicinal protein. The drug delivery carrier has an effect of efficiently loading medicinal proteins that are positively charged at a pH value of an isoelectric point or less of the medicinal proteins, based on electrostatic attraction. In addition, the drug delivery carrier releases medicinal proteins under human physiological conditions (pH 7.4, 37° C.) based on electrostatic repulsion and more specifically, has an effect of sustainedly releasing medicinal proteins for a long time between heat-sensitive polymer layers condensed due to body temperature.

### CROSS-REFERENCE TO RELATED APPLICATION

This U.S. non-provisional patent application claims priority under 35 U.S.C §119 of Korean Patent Application No. 10-2015-0141034, filed on Oct. 7, 2015, the entire contents of which are hereby incorporated by reference.

### SPECIFIC REFERENCE TO A GRACE PERIOD INVENTOR DISCLOSURE

This invention has been published in The Polymer Society of Korea Spring Meeting held from Apr. 8, 2015 to Apr. 10, 2015, and in the Journal of Applied Polymer Science (Human growth hormone-loaded nanogels composed of cinnamoyl alginate, cinnamoyl Pluronic F127, and cinnamoyl poly (ethylene glycol) on May 28,2015.

### BACKGROUND OF THE INVENTION

Field of the Invention

The present invention relates to a drug delivery carrier for sustained release of medicinal protein, and more particularly to a drug delivery carrier that allows medicinal protein positively charged at a pH level lower than an isoelectric point of the medicinal protein to be loaded in the drug delivery carrier, which is negatively charged, based on electrostatic attraction, and that, when administered to the human body, allows the medicinal protein negatively charged at a physiological pH higher than the isoelectric point of the medicinal protein to be detached and sustainedly released from the negatively charged drug delivery carrier, based on electrostatic repulsion, and a method for producing the same.

Description of the Related Art

Medicinal proteins such as insulin, interferon and erythropoietin (EPO) have large markets throughout the world. However, these medicinal proteins have a problem of difficulty in maintaining a constant level in the blood in the human body due to low stability and rapid metabolism or discharge.

For example, plasma levels of human growth hormones used for the treatment of growth retardation, Turner syndrome and chronic renal failure rapidly decrease, resulting in a problem of requiring daily subcutaneous injection (Salomon, Franco, et al. “The effects of treatment with recombinant human growth hormone on body composition and metabolism in adults with growth hormone deficiency.” New England Journal of Medicine 321.26 (1989): 1797-1803.). Accordingly, patient compliance with administration of human growth hormones is low and medical costs associated therewith are high.

In order to solve these problems, formulations having sustained actions by sustained release of human growth hormone for a long time were developed (Johnson, OluFunmi L., et al. “The stabilization and encapsulation of human growth hormone into biodegradable microspheres.” Pharmaceutical research 14.6 (1997): 730-735).

However, conventional sustained-acting formulations denature growth hormones during production and have a burst effect of initially releasing excessively high amounts of growth hormone, when administered to humans. As a result, the conventional formulations have drawbacks of insufficient sustained release and sustained action, and excessively high production costs.

Meanwhile, diabetes is a disease in which there are high blood glucose (sugar) levels due to reduced insulin secretion (Type I) or unrecognized insulin secretion (Type II). As symptoms become serious, diabetes involves tissue necrosis and complications, even death.

One treatment for Type I diabetes is administration of insulin by injection at an appropriate time every day. However, this treatment may cause shock due to rapid decrease in blood sugar levels immediately after administration and inevitably involves inconvenient and painful daily injection due to failure of long-term sustainment of active blood insulin levels.

To solve these drawbacks of injection, pulmonary delivery, transdermal delivery using microneedles, oral delivery and the like have been researched, but it is still difficult to control a single dose of insulin. In addition, problems of insulin denaturation by gastric digestive enzymes and insufficient sustained release and sustained action of insulin remain unsolved.

Accordingly, there is a need for approach and research on sustained release of medicinal proteins such as insulin in the human body.

### PRIOR ART DOCUMENT

### Patent Document

(Patent Document 1) Korean Patent No. 0949850 (registered on Mar. 19, 2010) discloses a temperature- and pH-sensitive block copolymer with excellent drug release profiles that contains (a) a polyethylene glycol-based compound (A); and (b) a poly(amido amine)-based oligomer (B), which are coupled to each other, a method for preparing the same and a hydrogel-type drug delivery carrier using the same.
(Patent Document 2) Korean Patent No. 1173608 (registered on Aug. 7, 2012) discloses a temperature- and pH-responsive dendrimer-linear polymer copolymer which exhibits considerably high drug filling rate upon use as a drug delivery carrier, and a drug delivery carrier or a biosensor containing the same.

### SUMMARY OF THE INVENTION

Therefore, the present invention has been made in view of the above problems, and it is an object of the present invention to provide a drug delivery carrier that loads medicinal proteins under acidic conditions based on electrostatic attraction, but detaches itself from the medicinal proteins under physiological conditions (pH 7.4, 37° C.) and sustainedly releases the medicinal proteins for a long time between heat-sensitive polymer layers condensed by body temperature.

In accordance with the present invention, the above and other objects can be accomplished by the provision of a drug delivery carrier including an alginate-cinnamic acid (Al-Ci) conjugate and a Pluronic F127-cinnamic acid (Pl-Ci) conjugate coupled to each other via cinnamic acid.

In accordance with another aspect of the present invention, provided is a drug delivery carrier including an alginate-cinnamic acid (Al-Ci) conjugate, a Pluronic F127-cinnamic acid (Pl-Ci) conjugate and a polyethylene glycol-cinnamic acid(PEG-Ci) conjugate coupled to one another via cinnamic acid.

Meanwhile, preferably, the coupling via the cinnamic acid may be carried out by dimerization of the cinnamic acid. In this case, the dimerization may be carried out by, for example, ultraviolet light.

Meanwhile, preferably, the drug delivery carrier may have a spherical shape.

Meanwhile, preferably, the drug delivery carrier may load protein. In this case, the protein may be for example medicinal protein. In addition, preferably, the protein is positively charged at a pH level lower than an isoelectric point, but is negatively charged at a pH level higher than the isoelectric point. In addition, the drug delivery carrier may load the protein at pH 2.0 to 4.0 and at 0 to 10° C. The drug delivery carrier may in vivo release the loaded protein to the outside. Preferably, the drug delivery carrier may sustainedly release the loaded protein.

Meanwhile, the alginate-cinnamic acid (Al-Ci) conjugate may be for example synthesized by condensation reaction of a carboxyl group of the cinnamic acid with a hydroxyl group of alginate. In addition, the Pluronic F127-cinnamic acid (Pl-Ci) conjugate may be for example synthesized by condensation reaction of a carboxyl group of the cinnamic acid with a hydroxyl group of Pluronic F127.

Meanwhile, the polyethylene glycol-cinnamic acid (PEG-Ci) conjugate may be for example synthesized by condensation reaction of a carboxyl group of the cinnamic acid with a hydroxyl group of polyethylene glycol.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows a principle in which medicinal protein is incorporated in a drug delivery carrier including an alginate-cinnamic acid conjugate (Al-Ci conjugate), a Pluronic F127-cinnamic acid conjugate (Pl-Ci conjugate) and a polyethylene glycol-cinnamic acid conjugate (PEG-Ci conjugate) according to the present invention, and a principle in which the loaded medicinal protein is released from the drug delivery carrier at a body temperature and at a body pH;

FIG. 2 is a ¹H NMR spectrum of the Al-Ci conjugate;

FIG. 3 is a ¹H NMR spectrum of the Pl-Ci conjugate;

FIG. 4 is a ¹H NMR spectrum of the PEG-Ci conjugate;

FIG. 5 shows measurement results of photo-dimerization of cinnamoyl residues with respect to an Al-Ci/Pl-Ci/PEG-Ci (1/1/1) drug delivery carrier (), an Al-Ci/Pl-Ci/PEG-Ci (1/1/0) drug delivery carrier (∇), an Al-Ci/Pl-Ci/PEG-Ci (1/0/1) drug delivery carrier (▪) and an Al-Ci/Pl-Ci/PEG-Ci (0/1/1) drug delivery carrier (⋄);

FIG. 6 shows TEM images of the Al-Ci/Pl-Ci/PEG-Ci (1/1/1) drug delivery carrier (A), the Al-Ci/Pl-Ci/PEG-Ci (1/1/0) drug delivery carrier (B), the Al-Ci/Pl-Ci/PEG-Ci (1/0/1) drug delivery carrier (C) and the Al-Ci/Pl-Ci/PEG-Ci (0/1/1) drug delivery carrier (D) after UV treatment for one four;

FIG. 7 shows results of zeta potentials measured according to variation in pH with respect to the UV-treated Al-Ci/Pl-Ci/PEG-Ci (1/1/1) drug delivery carrier (), the Al-Ci/Pl-Ci/PEG-Ci (1/1/0) drug delivery carrier (∇), the Al-Ci/Pl-Ci/PEG-Ci (1/0/1) drug delivery carrier (▪) and the Al-Ci/Pl-Ci/PEG-Ci (0/1/1) drug delivery carrier (⋄);

FIG. 8 shows results of zeta potentials of human grown hormone () and insulin (∇) measured at pH 3 to 9;

FIG. 9 shows results of release profiles of human growth hormone from the Al-Ci/Pl-Ci/PEG-Ci (1/1/1) drug delivery carrier observed at 37° C. and at pH 3.0 (), pH 5.0 (∇), pH 7.4 (▪), and pH 9.0 (⋄) for 170 hours;

FIG. 10 shows results of release profiles of human growth hormone from the Al-Ci/Pl-Ci/PEG-Ci (1/1/0) drug delivery carrier observed at 37° C. and at pH 3.0 (), pH 5.0 (∇), pH 7.4 (▪), and pH 9.0 (⋄) for 170 hours;

FIG. 11 shows results of release profiles of human growth hormone from the Al-Ci/Pl-Ci/PEG-Ci (1/0/1) drug delivery carrier observed at 37° C. and at pH 3.0 (), pH 5.0 (∇), pH 7.4 (▪), and pH 9.0 (⋄) for 170 hours;

FIG. 12 shows results of release profiles of insulin from the Al-Ci/Pl-Ci/PEG-Ci (1/1/1) drug delivery carrier observed at 37° C. and at pH 3.0 (), pH 5.0 (∇), pH 7.4 (▪), and pH 9.0 (⋄) for 170 hours;

FIG. 13 shows results of release profiles of insulin from the Al-Ci/Pl-Ci/PEG-Ci (1/1/0) drug delivery carrier observed at 37° C. and at pH 3.0 (), pH 5.0 (∇), pH 7.4 (▪), and pH 9.0 (⋄) for 170 hours; and

FIG. 14 shows results of release profiles of insulin from the Al-Ci/Pl-Ci/PEG-Ci (1/0/1) drug delivery carrier observed at 37° C. and at pH 3.0 (), pH 5.0 (∇), pH 7.4 (▪), and pH 9.0 (⋄) for 170 hours.

### DETAILED DESCRIPTION OF THE INVENTION

The present invention is directed to a drug delivery carrier developed using an alginate-cinnamic acid conjugate (Al-Ci conjugate), a Pluronic F127-cinnamic acid conjugate (Pl-Ci conjugate) and a poly(ethylene glycol)-cinnamic acid conjugate (PEG-Ci conjugate).

The Al-Ci, Pl-Ci and PEG-Ci conjugates are synthesized by condensation reaction of carboxyl groups of cinnamic acid (CA) with hydroxyl groups of polymers. Cinnamic acid has a phenyl group and thus produces an amphiphilic polymer when chemically conjugated to a water-soluble polymer.

Since hydrophobic interaction is present between cinnamoyl residues, when the Al-Ci, Pl-Ci and PEG-Ci conjugates are dispersed in an aqueous medium, the polymers form self-assemblies which may be used as drug delivery carriers.

The cinnamic acid may be dimerized upon exposure of ultraviolet light. Accordingly, when ultraviolet light is emitted to the drug delivery carrier including the Al-Ci conjugate, the Pl-Ci conjugate and the PEG-Ci conjugate, polymer chains constituting the drug delivery carrier, cinnamic acid may be chemically coupled by dimerization of the cinnamoyl residues.

Meanwhile, to efficiently incorporate medicinal protein in the drug delivery carrier, electrostatic attraction between the medicinal protein and the Al-Ci conjugate is used. When the pH value of the aqueous medium is maintained lower than the isoelectric point (pI) of the medicinal protein, the medicinal protein is positively charged and is thus conjugated to an alginate chain of the negatively-charged Al-Ci conjugate based on electrostatic attraction. In addition, when the temperature of the aqueous medium is maintained lower than a gelation temperature of the Pluronic F127, Pluronic F127 chains of the Pl-Ci conjugate expand, thus enabling the medicinal protein to easily contact the Al-Ci conjugate of the drug delivery carrier.

Meanwhile, when the drug delivery carrier in which the medicinal protein is incorporated under the conditions is injected into the human body, the medicinal protein is detached from the Al-Ci conjugate chain. This is because the medicinal protein is negatively charged since the pH value (pH of about 7.2 to 7.4) of human blood is higher than the isoelectric point of the medicinal protein, and is thus detached from the alginate chain of the Al-Ci conjugate based on electrostatic repulsion. In addition, since the body temperature is higher than the gelation temperature of the Pluronic F127 chain, the Pluronic F127 chains of the Pl-Ci conjugate are thermally contracted due to the body temperature to form a condensed layer which delays release of medicinal protein detached from the Pluronic F127 chain.

PEG of the PEG-Ci conjugate, which is a highly flexible polymer, performs a spring-like action to prevent human proteins from being adsorbed on the drug delivery carrier and thereby help the drug delivery carrier to circulate in the human circulation system for a long time.

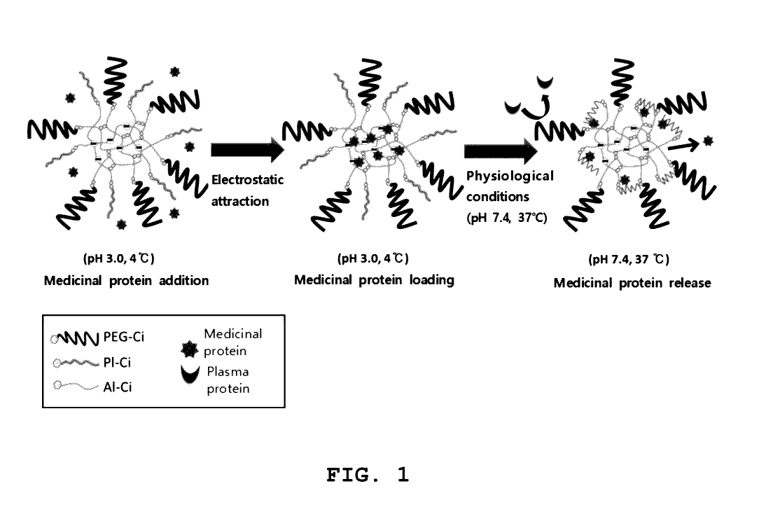

Meanwhile, FIG. 1 shows a principle in which medicinal protein is incorporated in the drug delivery carrier including the Al-Ci conjugate, the Pl-Ci conjugate and the PEG-Ci conjugate, and a principle in which the medicinal protein is released from the drug delivery carrier at a body temperature and at a body pH. A plurality of cinnamic acids (Ci) are conjugated to alginate (Al). When cinnamic acids are dimerized, alginates aggregate via the dimerized cinnamic acids. As such, when alginates aggregate, aggregates thereof form a core, and Pl and PEG face outward.

Hereinafter, the present invention will be described in more detail with reference to the following examples, and the scope of the present invention is not limited to the examples and includes variations of technical concepts equivalent thereto.

### EXAMPLE 1 AND COMPARATIVE EXAMPLES 1 to 3

### Production of Al-Ci Conjugate/Pl-Ci Conjugate/PEG-Ci Conjugate Drug Delivery Carrier

In this Example, an Al-Ci conjugate, a Pl-Ci conjugate and a PEG-Ci conjugate were respectively produced and a drug delivery carrier including the Al-Ci conjugate, the Pl-Ci conjugate and the PEG-Ci conjugate was produced using the same.

### <Production of Alginate-Cinnamic Acid Conjugate (Al-Ci Conjugate)>

1 g of alginate was dissolved in 17.5 ml of sulfonic acid solution (2.9% (w/v), solvent: dimethylsulfoxide) contained in a round flask (250 ml, 2-neck) to prepare an alginate solution and the flask was immersed in a 60° C. oil bath to heat the alginate solution.

Meanwhile, 1.8 g of cinnamoyl chloride was dissolved in 3 ml of a pyridine/dimethylsulfoxide mixed solvent (1/5, v/v). Then, the cinnamoyl chloride solution was added to the alginate solution and the mixed solution was stirred at 85° C. for 72 hours to induce a conjugation reaction.

Then, the reaction mixture was allowed to cool to room temperature and the product, Al-Ci conjugate was precipitated by addition of ethanol. Then, the Al-Ci conjugate was dissolved, reprecipitated and purified. Then, the purified Al-Ci conjugate was obtained by filtration and dried in a vacuum oven.

After drying, in order to verify synthesis of the Al-Ci conjugate, the dried Al-Ci conjugate was dissolved in D₂O and ¹H NMR spectrum was measured using a Bruker Avance 400 spectrometer (Karlsruhe, Germany, located in the Central Laboratory of Kangwon National University).

As a result of the measurement, it was seen that the signal observed at 3.7 to 4.1 ppm was a pyranose proton signal of alginate and the signal observed at 7.1 to 7.5 ppm was a cinnamoyl proton signal.

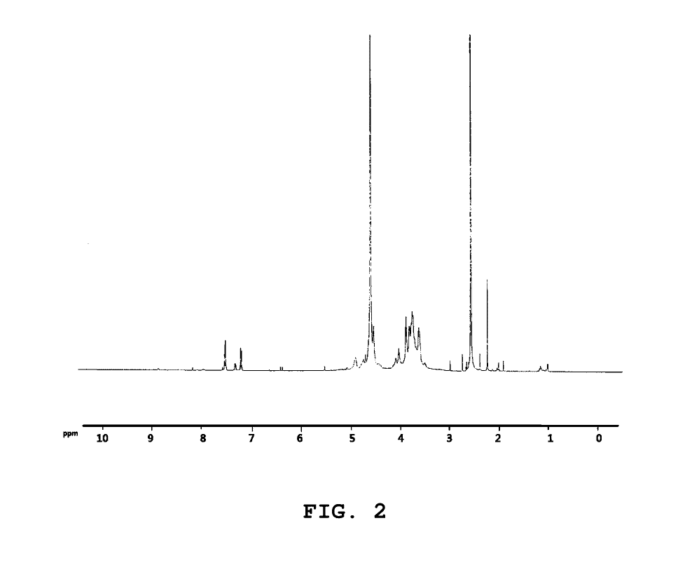

A molar ratio of pyranose unit of alginate to cinnamoyl residue was calculated using areas of the respective signals. It could be seen from the calculation result, the molar ratio was 1:0.25 and, upon conversion, one cinnamoyl residue was conjugated per four pyranose units (FIG. 2). FIG. 2 is a ¹H NMR spectrum of the Al-Ci conjugate.

### <Production of Pluronic F127-Cinnamic Acid Conjugate (Pl-Ci Conjugate)>

2.5 g of Pluronic F-127 was dissolved in 50 ml of dichloromethane contained in a 250 ml round bottom flask and 0.7 ml of triethylamine was subsequently dissolved therein to prepare a Pluronic F-127 solution.

0.13 g of cinnamoyl chloride was added to the Pluronic F-127 solution to obtain a reaction mixture and the reaction mixture was stirred in an ice bath for 12 hours and was additionally stirred at room temperature for 24 hours.

The product was precipitated by addition of diethylether to the reaction mixture and was filtered to obtain a Pl-Ci conjugate. To purify the Pl-Ci conjugate, the Pl-Ci conjugate was dissolved in dichloromethane and was reprecipitated by addition of diethylether.

The purified Pl-Ci conjugate was obtained by filtration and was dried in a vacuum oven. To verify synthesis of the Pl-Ci conjugate, the dried Pl-Ci conjugate was dissolved in dimethyl sulfoxide (DMSO-d₆) and ¹H NMR spectrum was measured using a Bruker Avance 400 spectrometer (Karlsruhe, Germany).

As a result of the measurement, it was seen that the signal of cinnamoyl proton was observed at 6.5 to 7.8 ppm and the signal of αCH—CO— of the cinnamoyl residue was observed at 6.5 ppm. The signals of aromatic proton of the cinnamoyl residue were observed at 7.5 ppm and 7.8 ppm, and the signal of —C—CH═ of the cinnamoyl residue was observed at 7.6 ppm.

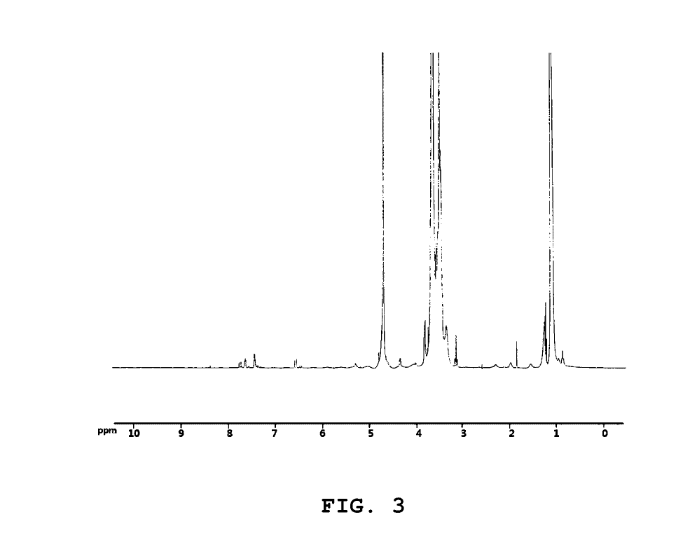

In addition, the signal of methyl proton of the poly(propylene oxide) block was observed at 1.05 ppm. A molar ratio of Pluronic F-127 to the cinnamoyl residue was calculated using areas of the respective signals. As a result, the molar ratio was 1:1.46 (FIG. 3). FIG. 3 is a ¹H NMR spectrum of the Pl-Ci conjugate.

### <Production of Polyethylene Glycol-Cinnamic Acid Conjugate (PEG-Ci Conjugate) >

2.5 g of polyethylene glycol was dissolved in 50 ml of dichloromethane contained in a 250 ml round bottom flask and 0.7 g of triethylamine was subsequently dissolved therein to prepare a polyethylene glycol solution.

0.15 g of cinnamoyl chloride was added to the polyethylene glycol solution to obtain a reaction mixture and the reaction mixture was stirred in an ice bath for 12 hours and was additionally stirred at room temperature for 24 hours.

After stirring, the product was precipitated by addition of the reaction mixture to diethylether and was filtered to obtain a PEG-Ci conjugate. To purify the PEG-Ci conjugate, the PEG-Ci conjugate was dissolved in dichloromethane and was reprecipitated by addition of diethylether.

The purified PEG-Ci conjugate was obtained by filtration and was dried in a vacuum oven. To verify synthesis of the PEG-Ci conjugate, the dried PEG-Ci conjugate was dissolved in CDCl₃ and ¹H NMR spectrum was measured using a Bruker Avance 400 spectrometer (Karlsruhe, Germany).

As a result of the measurement, it was seen that the proton signal of cinnamoyl residue was observed at 6.5 to 7.8 ppm and the signal of ethylene proton of PEG was observed at 3.7 ppm. A molar ratio of cinnamoyl residue to PEG chain was calculated using areas of the respective signals. As a result, the molar ratio was 1:1.50 (FIG. 4). FIG. 4 is a ¹H NMR spectrum of the PEG-Ci conjugate.

### <Mixing of Al-Ci Conjugate/Pl-Ci Conjugate/PEG-Ci Conjugate>

10 mg of the Al-Ci conjugate, 10 mg of the Pl-Ci conjugate and 10 mg of the PEG-Ci conjugate were dissolved in 3 ml of a glycine buffer solution (30 mM, pH 3.0) contained in a glass vial to adjust a weight ratio of the Al-Ci conjugate, the Pl-Ci conjugate and the PEG-Ci conjugate to 1:1:1.

In addition, 15 mg of the Al-Ci conjugate and 15 mg of the Pl-Ci conjugate were dissolved in 3 ml of a glycine buffer solution (30 mM, pH 3.0) contained in a glass vial to adjust a weight ratio of the Al-Ci conjugate, the Pl-Ci conjugate and the PEG-Ci conjugate to 1:1:0.

In addition, 15 mg of the Al-Ci conjugate and 15 mg of the PEG-Ci conjugate were dissolved in 3 ml of a glycine buffer solution (30 mM, pH 3.0) contained in a glass vial to adjust a weight ratio of the Al-Ci conjugate, the Pl-Ci conjugate and the PEG-Ci conjugate to 1:0:1.

In addition, 0 mg of the Al-Ci conjugate, 15 mg of the Pl-Ci conjugate and 15 mg of the PEG-Ci conjugate were dissolved in 3 ml of a glycine buffer solution (30 mM, pH 3.0) contained in a glass vial to adjust a weight ratio of the Al-Ci conjugate, the Pl-Ci conjugate and the PEG-Ci conjugate to 0:1:1.

The drug delivery carriers including the Al-Ci conjugate, the Pl-Ci conjugate and the PEG-Ci conjugate at weight ratios of 1:1:1, 1:1:0, 1:0:1 and 0:1:1 are simply referred to as “Al-Ci/Pl-Ci/PEG-Ci (1/1/1)”, “Al-Ci/Pl-Ci/PEG-Ci (1/1/0)”, “Al-Ci/Pl-Ci/PEG-Ci (1/0/1)”, and “Al-Ci/Pl-Ci/PEG-Ci (0/1/1)”, respectively.

To photo-dimerize CA residues of the Al-Ci/Pl-Ci/PEG-Ci drug delivery carriers having respective weight ratios, ultraviolet light was emitted to suspensions of the drug delivery carriers. Then, the Al-Ci/Pl-Ci/PEG-Ci (1/1/1), Al-Ci/Pl-Ci/PEG-Ci (1/1/0), Al-Ci/Pl-Ci/PEG-Ci (1/0/1) and Al-Ci/Pl-Ci/PEG-Ci (0/1/1) drug delivery carrier suspensions were diluted with distilled water to adjust concentrations of the CA residues to 0.05% (w/v) and 5 ml of each diluted drug delivery carrier suspension was charged in a glass vial. Then, UV (365 nm, 400 W) was vertically emitted to the glass vial with an open cover for one hour to obtain a photo-dimerized Al-Ci/Pl-Ci/PEG-Ci (1/1/1) drug delivery carrier (Example 1), Al-Ci/Pl-Ci/PEG-Ci (1/1/0) drug delivery carrier (Comparative Example 1), Al-Ci/Pl-Ci/PEG-Ci (1/0/1) drug delivery carrier (Comparative Example 2), and Al-Ci/Pl-Ci/PEG-Ci (0/1/1) drug delivery carrier (Comparative Example 3). After UV emission, dimerization was calculated in accordance with the following Equation 1:

Dimerization (%)=(1−Aₜ/Aₒ)×100% [Equation 1]

In Equation 1, A₀ represents absorbance of drug delivery carrier suspension at 275 nm before UV emission, and Aₜ represents absorbance of drug delivery carrier suspension at 275 nm after UV emission for a predetermined time.

As a result of measurement, the dimerization increased in the form of a saturated curve. The CA dimers produced by UV emission might be photo-degraded by UV emission and converted into monomers. Accordingly, it could be seen that an equilibrium at which the photo-dimerization rate was equal to the photo-degradation rate existed, as UV emission time passed.

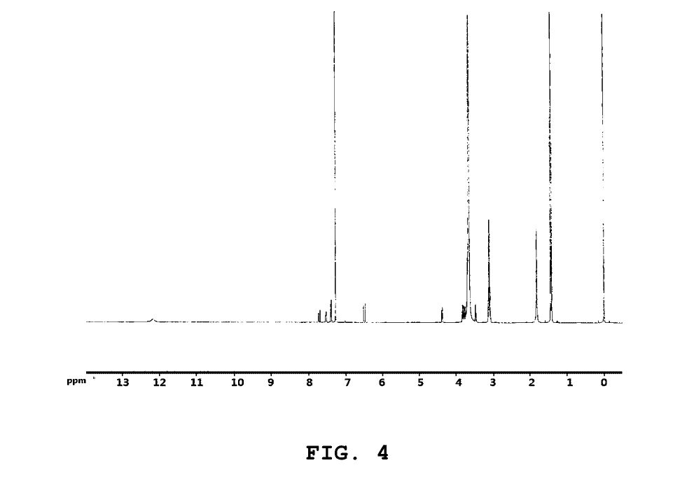

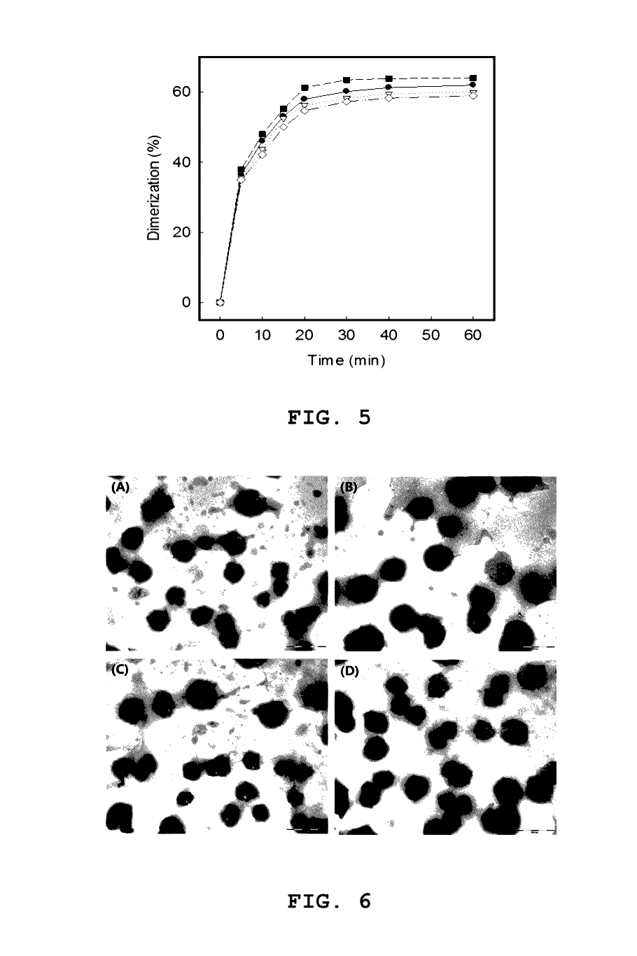

Accordingly, it could be seen that the dimerization rapidly increased in initial UV emission and then slowly increased as UV emission time passed (FIG. 5). FIG. 5 shows measurement results of photo-dimerization of CA residues of Al-Ci/Pl-Ci/PEG-Ci (1/1/1), Al-Ci/Pl-Ci/PEG-Ci (1/1/0), Al-Ci/Pl-Ci/PEG-Ci (1/0/1) and Al-Ci/Pl-Ci/PEG-Ci (0/1/1) drug delivery carriers.

### EXPERIMENTAL EXAMPLE 1

### TEM of Drug Delivery Carriers Including Al-Ci Conjugate/Pl-Ci Conjugate/PEG-Ci Conjugate

In the present experimental example, the cross-sectional structures of the Al-Ci/Pl-Ci/PEG-Ci drug delivery carriers were observed.

For this purpose, the Al-Ci/Pl-Ci/PEG-Ci (1/1/1), Al-Ci/Pl-Ci/PEG-Ci (1/1/0), Al-Ci/Pl-Ci/PEG-Ci (1/0/1) and Al-Ci/Pl-Ci/PEG-Ci (0/1/1) drug delivery carriers were stained with phosphotungstic acid and images thereof were obtained with a transmission electron microscope (TEM) (LEO 912AB OMEGA, Germany, installed in Korea Basic Science Institute, Chuncheon, Republic of Korea).

As a result of TEM, the drug delivery carriers were found to have a diameter of 50 to 150 nm and a substantially spherical shape. Cinnamic acid is a hydrophobic compound having a phenyl group, whereas alginate, Pluronic F127 and polyethylene glycol are hydrophilic polymers and the Al-Ci conjugate, the Pl-Ci conjugate and the PEG-Ci conjugate are thus amphiphilic.

Accordingly, it was analyzed that these conjugates were associated in an aqueous medium by hydrophobic interaction of cinnamoyl residues to form particulate drug delivery carriers such as polymeric micelles.

### EXPERIMENTAL EXAMPLE 2

### Measurement of Zeta Potentials of Drug Delivery Carriers and Medicinal Proteins

In the present experimental example, zeta potentials of the Al-Ci/Pl-Ci/PEG-Ci drug delivery carriers, and human grown hormone and insulin as medicinal proteins were measured.

The surface charges of the Al-Ci/Pl-Ci/PEG-Ci (1/1/1), Al-Ci/Pl-Ci/PEG-Ci (1/1/0), Al-Ci/Pl-Ci/PEG-Ci (1/0/1) and Al-Ci/Pl-Ci/PEG-Ci (0/1/1) drug delivery carriers were measured using a dynamic light scattering equipment (ZetaPlus 90, Brookhaven Instrument Co, New York, USA) at room temperature with a variation in pH from 3.0 to 9.0.

Buffer was used to vary the pH value. Glycine buffer was used to adjust pH to 3.0, 4.0, or 9.0, and MES buffer was used to adjust pH to 5.0 or 6.0. PBS buffer was used to adjust pH to 7.0 or 8.0.

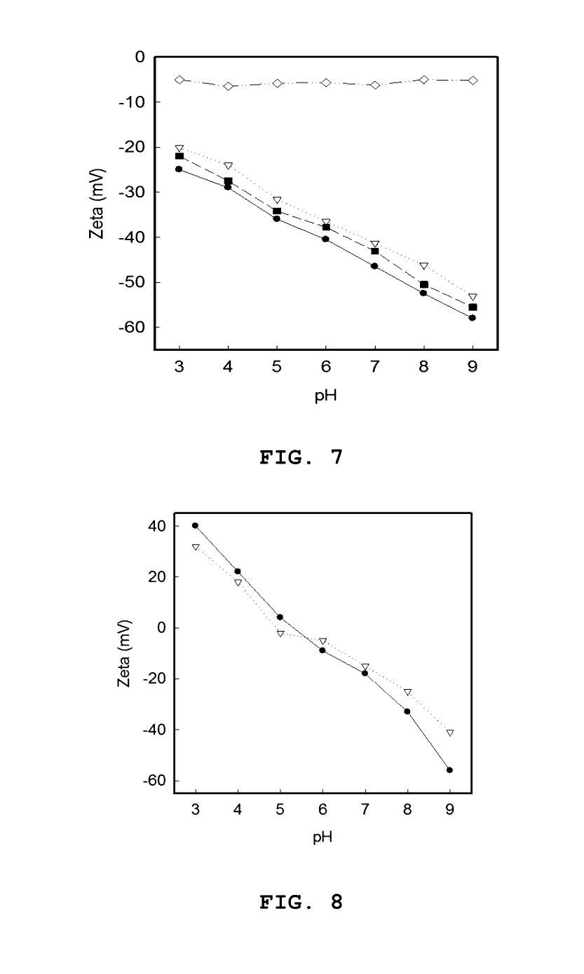

As a result of the test, zeta potentials of all the drug delivery carriers were negative values in the measured pH range. Absolute values of zeta potentials of the drug delivery carriers, except for the Al-Ci/Pl-Ci/PEG-Ci (0/1/1) drug delivery carrier, increased with increase in pH. Since carboxylic acid of alginate has an increased ionization with an increase in pH, absolute values of zeta potentials of alginate-containing drug delivery carriers {Al-Ci/Pl-Ci/PEG-Ci (1/1/1), Al-Ci/Pl-Ci/PEG-Ci (1/1/0) and Al-Ci/Pl-Ci/PEG-Ci (1/0/1) drug delivery carriers} increased with an increase in pH. Since the Al-Ci/Pl-Ci/PEG-Ci (0/1/1) drug delivery carrier contained no alginate, the zeta potential was almost constant regardless of variation in pH and the absolute value thereof was only about 5 (FIG. 7). FIG. 7 shows results of zeta potentials measured according to variation in pH with respect to UV-treated Al-Ci/Pl-Ci/PEG-Ci (1/1/1), Al-Ci/Pl-Ci/PEG-Ci (1/1/0), Al-Ci/Pl-Ci/PEG-Ci (1/0/1) and Al-Ci/Pl-Ci/PEG-Ci (0/1/1) drug delivery carriers.

Meanwhile, as pH increased from 3.0 to 9.0, zeta potential of human grown hormone decreased from +39 mV to −51 mV, the isoelectric point was observed at pH of about 5.0, whereas the zeta potential of insulin decreased from +31 mV to −42 mV and the isoelectric point was observed at pH of about 5.0 (FIG. 8). FIG. 8 shows results of zeta potentials of human grown hormone and insulin measured at pH 3 to 9.

### EXMPLE 2

### Incorporation of Medicinal Proteins in Drug Delivery Carriers

In the present example, medicinal proteins were incorporated in the Al-Ci/Pl-Ci/PEG-Ci drug delivery carriers.

5 mg of human growth hormone was dissolved in 1 ml of a glycine buffer solution (30 mM, pH 3.0). Then, 5 mg of insulin was dissolved in 1 ml of a glycine buffer solution (30 mM, pH 3.0). 1 ml of the medicinal protein solution was added to an Al-Ci/Pl-Ci/PEG-Ci drug delivery carrier suspension (1%, w/v) to prepare a mixture.

Then, the mixture was stirred at 4° C. for 24 hours, and to remove the residual medicinal proteins not-incorporated in the drug delivery carrier, 4 ml of the mixture (drug delivery carrier+medicinal protein) was charged in a dialysis bag (MWCO 100,000) and dialysis was performed using 500 ml of a glycine buffer solution (30 mM, pH 3.0) until the medicinal proteins were not released any more.

In the case of human grown hormone, respective specific loadings (percentage of weight of human growth hormone loaded with respect to weight of the drug delivery carrier) of the Al-Ci/Pl-Ci/PEG-Ci (1/1/1), Al-Ci/Pl-Ci/PEG-Ci (1/1/0), Al-Ci/Pl-Ci/PEG-Ci (1/0/1) and Al-Ci/Pl-Ci/PEG-Ci (0/1/1) drug delivery carriers were 3.3% (w/w), 4.5% (w/w), 4.3% (w/w), and 0.1% (w/w) in this order.

The principle in which human growth hormone is loaded in the alginate-containing drug delivery carriers {Al-Ci/Pl-Ci/PEG-Ci (1/1/1), Al-Ci/Pl-Ci/PEG-Ci (1/1/0) and Al-Ci/Pl-Ci/PEG-Ci (1/0/1)} was considered based on the facts that human growth hormone is negatively charged at pH of 3 (see FIG. 8) and is thus adsorbed on the negatively charged drug delivery carrier (see FIG. 7) through electrostatic attraction.

On the other hand, the specific loading of the alginate-free drug delivery carrier {Al-Ci/Pl-Ci/PEG-Ci (0/1/1) drug delivery carrier} was merely 0.1% because human grown hormone was not efficiently absorbed on the drug delivery carrier due to small negative absolute value of zeta potential of the drug delivery carrier (see FIG. 7, about 4 mV).

Meanwhile, in the case of insulin, respective specific loadings of the Al-Ci/Pl-Ci/PEG-Ci (1/1/1), Al-Ci/Pl-Ci/PEG-Ci (1/1/0), Al-Ci/Pl-Ci/PEG-Ci (1/0/1) and Al-Ci/Pl-Ci/PEG-Ci (0/1/1) drug delivery carriers (percentage of weight of insulin loaded with respect to weight of the drug delivery carrier) were 3.3% (w/w), 4.5% (w/w), 4.3% (w/w) and 0.1% (w/w) in this order. The principle in which insulin is loaded in the alginate-containing drug delivery carriers {Al-Ci/Pl-Ci/PEG-Ci (1/1/1), Al-Ci/Pl-Ci/PEG-Ci (1/1/0) and Al-Ci/Pl-Ci/PEG-Ci (1/0/1)} was considered based on the facts that insulin is negatively charged at pH of 3 (see FIG. 8) and is thus adsorbed on the negatively charged drug delivery carrier (see FIG. 7) through electrostatic attraction.

On the other hand, the specific loading of the alginate-free drug delivery carrier {Al-Ci/Pl-Ci/PEG-Ci (0/1/1) drug delivery carrier} was merely 0.1% because insulin was not efficiently absorbed on the drug delivery carrier due to small negative absolute value of zeta potential of the drug delivery carrier (see FIG. 7, about 4 mV).

### EXPERIMENTAL EXAMPLE 3

### Release of Human Growth Hormone from Drug Delivery Carriers

In the present experimental example, release levels of medicinal proteins from the drug delivery carriers having loaded medicinal proteins, prepared in Example 5 were measured according to pH and time.

1 ml of each of suspensions {1% (w/v), glycine buffer solution (pH 3.0)} of the human growth hormone-loaded Al-Ci/Pl-Ci/PEG-Ci (1/1/1), Al-Ci/Pl-Ci/PEG-Ci (1/1/0) and Al-Ci/Pl-Ci/PEG-Ci (1/0/1) drug delivery carriers was charged in a dialysis bag (MWCO 100,000), followed by sealing. The suspension was dialyzed in 40 ml of a 37° C. buffer solution (pH 3.0, pH 5.0, pH 7.4 or pH 9.0) contained in a 50 ml beaker.

0.2 ml of the dialyzed solution was collected at a certain time for 7 days, the dialysis period, to measure the weight of the released human growth hormone. The amount of human growth hormone was measured using a kit for assay of human growth hormone (R&D System Elisa).

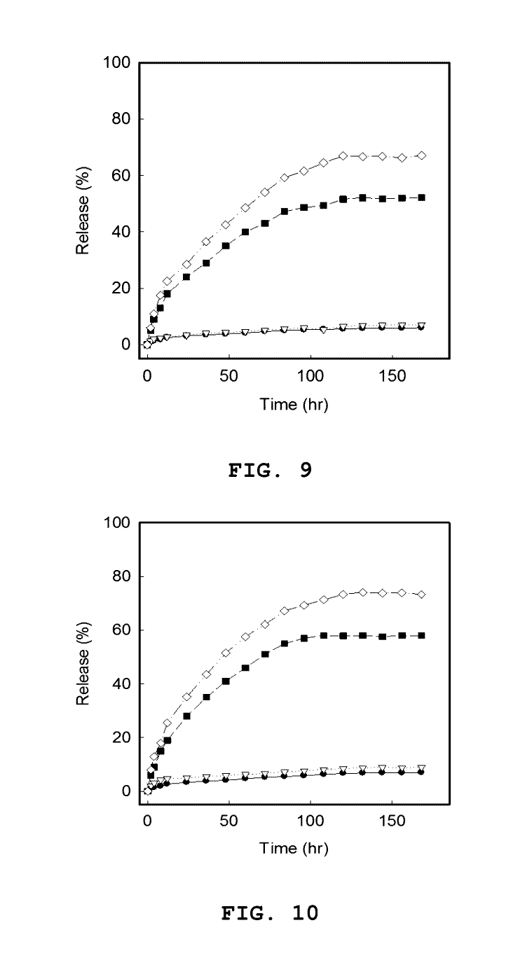

As a result of the test, the Al-Ci/Pl-Ci/PEG-Ci (1/1/1) drug delivery carrier had a very low release rate at pH levels of the release medium of 3.0 and 5.0 and a 170-hour release level lower than 10%. However, when the pH of the release medium was 7.4, the release level continuously increased for 170 hours and the 170 hour-release level was about 52.1%. When the pH of the release medium was 9.0, the release level continuously increased for 170 hours and the 170 hour-release level was about 67.2%. It was considered that the release level was low under an acidic condition and was high at pH levels of 7.4 and 9 because the human growth hormone is positively charged in the acidic condition and is thus electrostatically attracted to the negatively charged Al-Ci/Pl-Ci/PEG-Ci (1/1/1) drug delivery carrier, whereas the human growth hormone is negatively charged at pH levels of 7.4 and 9, and thus cannot be electrostatically attracted to the negatively charged Al-Ci/Pl-Ci/PEG-Ci (1/1/1) drug delivery carrier (FIG. 9). FIG. 9 shows results of release profiles of human growth hormone from the Al-Ci/Pl-Ci/PEG-Ci (1/1/1) drug delivery carrier observed at 37° C. and at pH levels of 3.0, 5.0, 7.4 and 9.0 for 170 hours.

Meanwhile, the Al-Ci/Pl-Ci/PEG-Ci (1/1/0) drug delivery carrier had a very low release rate at pH levels of the release medium of 3.0 and 5.0 and a 170-hour release level lower than 10%. However, when the pH of the release medium was 7.4, the release level continuously increased for 170 hours and the 170 hour-release level was about 57.6%. When the pH of the release medium was 9.0, the release level continuously increased for 170 hours and the 170 hour-release level was about 73.5% (FIG. 10). FIG. 10 shows results of release profiles of human growth hormone from the Al-Ci/Pl-Ci/PEG-Ci (1/0/1) drug delivery carrier observed at 37° C. and at pH levels of 3.0, 5.0, 7.4 and 9.0 for 170 hours.

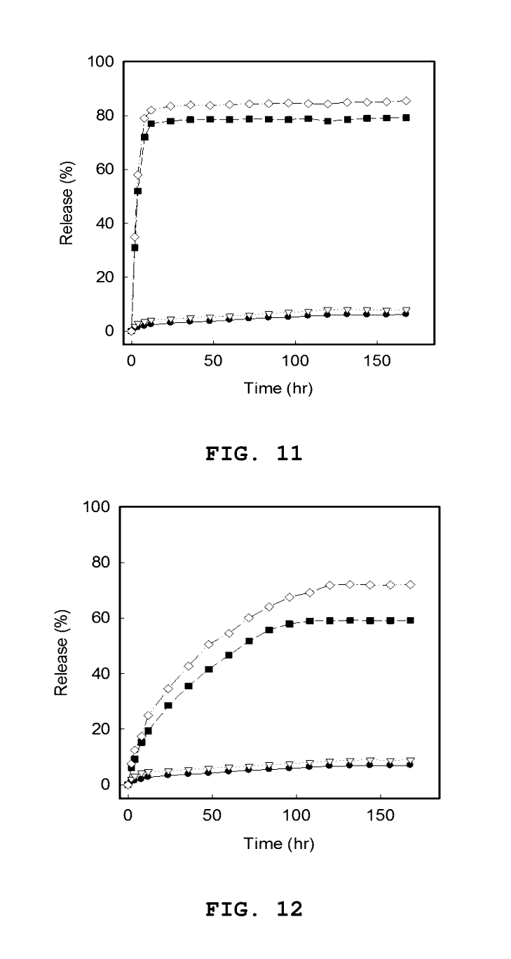

Meanwhile, the Al-Ci/Pl-Ci/PEG-Ci (1/0/1) drug delivery carrier had a very low release rate at pH levels of the release medium of 3.0 and 5.0 and a 170-hour release level lower than 10%. However, when the pH of the release medium was 7.4, the release level continuously increased for 170 hours and the 170 hour-release level was about 79.7%. When the pH of the release medium was 9.0, the release level continuously increased for 170 hours and the 170 hour-release level was about 85.5% (FIG. 11). FIG. 11 shows results of release profiles of human growth hormone from the Al-Ci/Pl-Ci/PEG-Ci (1/0/1) drug delivery carrier observed at 37° C. and at pH levels of 3.0, 5.0, 7.4 and 9.0 for 170 hours.

From the results, it could be seen that the Pl-Ci-containing drug delivery carriers {Al-Ci/Pl-Ci/PEG-Ci (1/1/1) drug delivery carrier and Al-Ci/Pl-Ci/PEG-Ci (1/1/0) drug delivery carrier) slowly sustainedly released medicinal proteins under human physiological conditions (37° C., pH 7.4) over 170 hours, whereas the Pl-Ci-free drug delivery carrier (Al-Ci/Pl-Ci/PEG-Ci (1/0/1)) rapidly released human growth hormone under human physiological conditions (37° C., pH 7.4) for a short time (10 hours).

As such, it was considered that the Pl-Ci-containing drug delivery carriers exhibited sustained release over a long time because Pluronic chains of Pl-Ci thermally condensed at 37° C. to form a membrane and the formed control membrane functioned as a control membrane for release of human growth hormone.

### EXPERIMENTAL EXAMPLE 4

### Release of Insulin From Drug Delivery Carriers

1 ml of suspensions (1% (w/v), glycine buffer solution (pH 3.0)) of the insulin-loaded Al-Ci/Pl-Ci/PEG-Ci (1/1/1), Al-Ci/Pl-Ci/PEG-Ci (1/1/0) and Al-Ci/Pl-Ci/PEG-Ci (1/0/1) drug delivery carriers was charged in a dialysis bag (MWCO 100,000), followed by sealing. The suspension was dialyzed in 40 ml of a 37° C. buffer solution (pH 3.0, pH 5.0, pH 7.4, pH 9.0) contained in a 50 ml beaker.

0.2 ml of the dialyzed solution was collected at a certain time for 7 days, the dialysis period to measure the weight of the released insulin. The amount of insulin was measured using an insulin assay kit (Arbor Assays).

As a result of the test, the Al-Ci/Pl-Ci/PEG-Ci (1/1/1) drug delivery carrier had a very low release rate at pH levels of the release medium of 3.0 and 5.0 and a 170-hour release level lower than 10%. However, when the pH of the release medium was 7.4, the release level continuously increased for 170 hours and the 170 hour-release level was about 59.1%. When the pH of the release medium was 9.0, the release level continuously increased for 170 hours and the 170 hour-release level was about 72.4% (FIG. 12). FIG. 12 shows results of release profiles of insulin from the Al-Ci/Pl-Ci/PEG-Ci (1/1/1) drug delivery carrier observed at 37° C. and at pH levels of 3.0, 5.0, 7.4 and 9.0 for 170 hours.

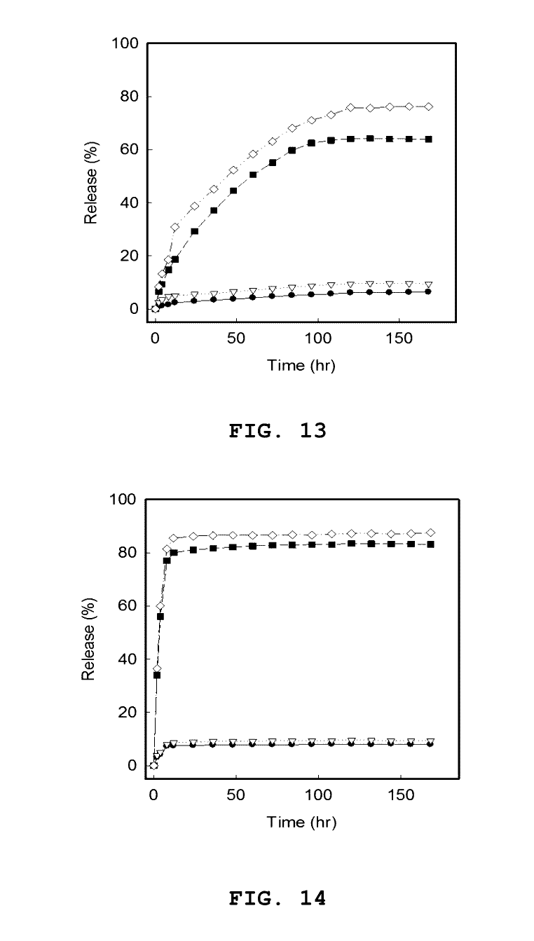

Meanwhile, the Al-Ci/Pl-Ci/PEG-Ci (1/1/0) drug delivery carrier had a very low release rate at pH levels of the release medium of 3.0 and 5.0 and a 170-hour release level lower than 10%. However, when the pH of the release medium was 7.4, the release level continuously increased for 170 hours and the 170 hour-release level was about 64.1%. When the pH of the release medium was 9.0, the release level continuously increased for 170 hours and the 170 hour-release level was about 76.2% (FIG. 13). FIG. 13 shows results of release profiles of insulin from the Al-Ci/Pl-Ci/PEG-Ci (1/1/0) drug delivery carrier observed at 37° C. and at pH levels of 3.0, 5.0, 7.4 and 9.0 for 170 hours.

Meanwhile, the Al-Ci/Pl-Ci/PEG-Ci (1/0/1) drug delivery carrier had a very low release rate at pH levels of the release medium of 3.0 and 5.0 and a 170-hour release level lower than 10%. However, when the pH of the release medium was 7.4, the release level rapidly increased for the first 10 hours and the 170 hour-release level was about 83.3%. When the pH of the release medium was 9.0, the release level rapidly increased for initial 10 hours and the 170 hour-release level was about 87.9% (FIG. 14). FIG. 14 shows results of release profiles of insulin from the Al-Ci/Pl-Ci/PEG-Ci (1/0/1) drug delivery carrier observed at 37° C. and at pH levels of 3.0, 5.0, 7.4 and 9.0 for 170 hours.

From the results, it could be seen that the Pl-Ci-containing drug delivery carriers {Al-Ci/Pl-Ci/PEG-Ci (1/1/1) drug delivery carrier and Al-Ci/Pl-Ci/PEG-Ci (1/1/0) drug delivery carrier} slowly sustainedly released insulin under human physiological conditions (37° C., pH 7.4) over 170 hours, whereas the Pl-Ci-free drug delivery carrier {Al-Ci/Pl-Ci/PEG-Ci (1/0/1)} rapidly release insulin under human physiological conditions (37° C., pH 7.4) for a short time (10 hours).

As such, it was considered that the Pl-Ci-containing drug delivery carriers exhibited sustained release over a long time because Pluronic chains of Pl-Ci thermally condensed at 37° C. to form a membrane and the formed membrane functioned as a control membrane for release of insulin.

As apparent from the above description, the drug delivery carrier according to the present invention has an effect of efficiently loading medicinal proteins that are positively charged at a pH value of an isoelectric point or less of the medicinal proteins, based on electrostatic attraction. In addition, the drug delivery carrier releases medicinal proteins under human physiological conditions (pH 7.4, 37° C.) based on electrostatic repulsion and more specifically, has an effect of sustainedly releasing medicinal proteins for a long time between heat-sensitive polymer layers condensed by body temperature.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A drug delivery carrier comprising an alginate-cinnamic acid (Al-Ci) conjugate and a Pluronic F127-cinnamic acid (Pl-Ci) conjugate coupled to each other via cinnamic acid.

2. The drug delivery carrier according to claim 1, wherein the coupling via the cinnamic acid is carried out by dimerization of the cinnamic acid.

3. The drug delivery carrier according to claim 2, wherein the dimerization is carried out by ultraviolet light.

4. The drug delivery carrier according to claim 1, wherein the drug delivery carrier loads protein which is medicinal protein.

5. The drug delivery carrier according to claim 4, wherein the protein is positively charged at a pH level lower than an isoelectric point, but is negatively charged at a pH level higher than the isoelectric point.

6. The drug delivery carrier according to claim 4, wherein the drug delivery carrier loads the protein at pH 2.0 to 4.0 and at 0 to 10° C.

7. The drug delivery carrier according to claim 4, wherein the drug delivery carrier in vivo sustainedly releases the loaded protein to the outside.

8. The drug delivery carrier according to claim 1, wherein the alginate-cinnamic acid (Al-Ci) conjugate is synthesized by condensation reaction of a carboxyl group of the cinnamic acid with a hydroxyl group of alginate.

9. The drug delivery carrier according to claim 1, wherein the Pluronic F127-cinnamic acid (Pl-Ci) conjugate is synthesized by condensation reaction of a carboxyl group of the cinnamic acid with a hydroxyl group of Pluronic F127.

10. A drug delivery carrier comprising an alginate-cinnamic acid (Al-Ci) conjugate, a Pluronic F127-cinnamic acid (Pl-Ci) conjugate and a polyethylene glycol-cinnamic acid(PEG-Ci) conjugate coupled to one another via cinnamic acid.

11. The drug delivery carrier according to claim 10, wherein the coupling via the cinnamic acid is carried out by dimerization of the cinnamic acid.

12. The drug delivery carrier according to claim 10, wherein the dimerization is carried out by ultraviolet light.

13. The drug delivery carrier according to claim 10, wherein the drug delivery carrier loads protein which is medicinal protein.

14. The drug delivery carrier according to claim 13, wherein the protein is positively charged at a pH level lower than an isoelectric point, but is negatively charged at a pH level higher than the isoelectric point.

15. The drug delivery carrier according to claim 13, wherein the drug delivery carrier loads the protein at pH 2.0 to 4.0 and at 0 to 10° C.

16. The drug delivery carrier according to claim 13, wherein the drug delivery carrier in vivo sustainedly releases the loaded protein to the outside.

17. The drug delivery carrier according to claim 10, wherein the alginate-cinnamic acid (Al-Ci) conjugate is synthesized by condensation reaction of a carboxyl group of the cinnamic acid with a hydroxyl group of alginate.

18. The drug delivery carrier according to claim 10, wherein the Pluronic F127-cinnamic acid (Pl-Ci) conjugate is synthesized by condensation reaction of a carboxyl group of the cinnamic acid with a hydroxyl group of Pluronic F127.

19. The drug delivery carrier according to claim 10, wherein the polyethylene glycol-cinnamic acid (PEG-Ci) conjugate is synthesized by condensation reaction of a carboxyl group of the cinnamic acid with a hydroxyl group of polyethylene glycol.
